(19) United States
(12) Patent Application Publication
(10) Pub. No.: US 2024/0090955 A1
(43) Pub. Date: Mar. 21, 2024

(54) SYSTEMS AND METHODS FOR REAL-TIME IMAGE-BASED DEVICE LOCALIZATION

(71) Applicant: NeuWave Medical, Inc., Madison, WI (US)

(72) Inventors: Kevin Royalty, Madison, WI (US); Louis Mingione, Madison, WI (US); Jeffrey Bissing, Madison, WI (US)

(21) Appl. No.: 18/521,080

(22) Filed: Nov. 28, 2023

Related U.S. Application Data
(62) Division of application No. 17/482,004, filed on Sep. 22, 2021.

(51) Int. Cl. A61B 34/20; A61B 6/12; A61B 90/00

(52) U.S. Cl. CPC A61B 34/20; A61B 6/12; A61B 90/39; A61B 2034/2051; A61B 2090/3966

(57) ABSTRACT

The present invention relates to flexible sheath assemblies capable of being localized in three-dimensions (i.e., determining the location and orientation) in real-time based on two-dimensional x-ray images, and related systems and methods.

### CROSS-REFERENCE TO RELATED APPLICATION

The present application is a divisional of U.S. patent application Ser. No. 17/482,004, filed Sep. 22, 2021.

### FIELD OF INVENTION

The present invention relates to flexible sheath assemblies and, more particularly, medical catheters and methods of locating medical catheters within a subject.

### BACKGROUND

Sheaths or catheters (e.g., an endoscopic sheath) needs to be flexible in order to navigate through peripheral locations that may include tortuous paths. Conventional catheters and endoluminal devices are difficult to localize in three-dimensional space using X-ray imaging, because the X-ray imaging only provides a two-dimensional view.

### SUMMARY

The present invention relates to flexible sheath assemblies capable of being localized in three-dimensions (i.e., determining the location and orientation) in real-time based on two-dimensional x-ray images, and related systems and methods.

In one aspect, the present disclosure provides a flexible sheath for use in medical procedures. The flexible sheath includes an elongate tubular body having an elongate tubular body proximal end and an elongate tubular body distal end. The flexible sheath further includes a first fiducial positioned at the elongate tubular body proximal end and a second fiducial spaced apart from the first fiducial. The first fiducial and the second fiducial provide visual X-ray indication of the location of the flexible sheath in three-dimensional space.

In some embodiments, the first fiducial and the second fiducial include a radiopaque material.

In some embodiments, the flexible sheath further includes a third fiducial, wherein the second fiducial is positioned between the first fiducial and the third fiducial.

In some embodiments, the first fiducial, the second fiducial, and the third fiducial are spaced an equal distance apart from each other.

In some embodiments, the flexible sheath further includes an asymmetric tip marker aligned to an articulation axis of the flexible sheath.

In some embodiments, the asymmetric tip marker includes a radiopaque material.

In some embodiments, the first fiducial is circular.

In some embodiments, an outer diameter of the first fiducial is equal to an outer diameter of the elongate tubular body.

In some embodiments, a thickness of the first fiducial is equal to a wall thickness of the elongate tubular body.

In another aspect, the present disclosure provides a flexible sheath for use in medical procedures. The flexible sheath includes an elongate tubular body having an elongate tubular body proximal end and an elongate tubular body distal end, and an asymmetrical tip marker positioned at the elongate tubular body distal end. The asymmetrical tip provides visual X-ray indication of the orientation of the elongate tubular body distal end in three-dimensional space.

In some embodiments, the asymmetrical tip includes a first longitudinal mark, a second longitudinal mark circumferentially spaced from the first longitudinal mark, and a third longitudinal mark circumferentially spaced from the second longitudinal mark. The second longitudinal mark is circumferentially positioned between the first longitudinal mark and the third longitudinal mark.

In some embodiments, the second longitudinal mark is longer than the first longitudinal mark and the third longitudinal mark.

In some embodiments, the first longitudinal mark is positioned closer to the elongate tubular body distal end than the third longitudinal mark.

In some embodiments, the flexible sheath further includes a first fiducial positioned at the elongate tubular body proximal end and a second fiducial spaced apart from the first fiducial. The first fiducial and the second fiducial provide visual X-ray indication of the location of the flexible sheath in three-dimensional space.

In another aspect, the present disclosure provides a method of localizing a flexible sheath in three-dimensional space. The method includes positioning the flexible sheath with at least one fiducial in an x-ray imaging system; capturing a two-dimensional x-ray image of the flexible sheath; identifying the at least one fiducial in the two-dimensional x-ray image; and determining an estimated location of the flexible sheath based on a geometric transform of the x-ray imaging system.

In some embodiments, determining the estimated location of the flexible sheath is further based on three-dimensional anatomical constraints of a patient.

In some embodiments, determining the estimated location of the flexible sheath is further based on a mechanical property of the flexible sheath.

In some embodiments, the method further includes validating the estimated location of the flexible sheath by reprojecting the estimated location of the at least one fiducial into a two-dimensional validation image, and calculating an error between the location of the at least one fiducial in the two-dimensional x-ray image and the two-dimensional validation image.

In some embodiments, determining of the estimated location is repeated until the error is below a threshold.

In some embodiments, the method includes displaying the estimated location of the flexible sheath in real-time.

In some embodiments, the method further includes determining an estimated orientation of the flexible sheath based on the at least one fiducial.

Additional embodiments are described herein.

### BRIEF DESCRIPTION OF THE DRAWINGS

The accompanying figures and examples are provided by way of illustration and not by way of limitation. The foregoing aspects and other features of the disclosure are explained in the following description, taken in connection with the accompanying example figures (also “FIG.”) relating to one or more embodiments.

FIG. 1A is a perspective view of a flexible sheath in a first location and orientation.

FIG. 1B is a perspective view of the flexible sheath of FIG. 1A in a second location and orientation.

FIG. 1C is a perspective view superimposing FIG. 1A and FIG. 1B together.

FIG. 2 is a view of a flexible sheath positioned in an x-ray imaging system.

FIG. 3 is a perspective view of a flexible sheath with a plurality of fiducials.

FIG. 4 is an enlarged partial view of FIG. 3, with a partial tear-away of the flexible sheath cross-section.

FIG. 5 is a schematic of a fiducial in the flexible sheath of FIG. 3 shown in two different side perspectives.

FIG. 6 is a method of localizing a flexible sheath.

FIG. 7A is a perspective view of a flexible sheath in a first location and orientation.

FIG. 7B is a perspective view of the flexible sheath of FIG. 7A in a second location and orientation.

FIG. 7C is a perspective view superimposing FIG. 7A and FIG. 7B together.

FIG. 8 is a perspective view of a flexible sheath including an asymmetrical tip marker.

FIG. 9 is a side view of the flexible sheath of FIG. 8 in a first orientation, with articulation occurring within the view plane and illustrated with arrows.

FIG. 10 is an enlarged partial view of FIG. 9.

FIG. 11 is a side view of the flexible sheath of FIG. 8 in a second orientation, with articulation occurring obliquely to the view plane.

FIG. 12 is an enlarged partial view of FIG. 11.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Therapeutic endoscopy or interventional endoscopy pertains to an endoscopic procedure during which a treatment (e.g., tissue ablation) (e.g., tissue collection) is carried out via the endoscope. This contrasts with diagnostic endoscopy, where the aim of the procedure is purely to visualize an internal part of a body (e.g., gastrointestinal region, respiratory region, urinary tract region, etc.) in order to aid diagnosis. In practice, a procedure which starts as a diagnostic endoscopy may become a therapeutic endoscopy depending on the findings.

Generally, therapeutic endoscopy involves the administration of an endoscope (“primary catheter”) into a body region until a natural stopping positioning is reached (e.g., until the circumference of the body region inhibits further advancement of the endoscope). Next, a flexible sheath having a circumference smaller than the circumference of the endoscope is advanced through the endoscope and to a desired body region location. Next, a therapeutic or diagnostic tool (e.g., an ablation energy delivery tool) (e.g., a tissue collection tool) (e.g., biopsy needle) having a circumference smaller than the diameter of the flexible sheath is advanced through the flexible sheath to the desired body region location. Next, ablation energy is delivered to the desired body region location. Upon completion of the therapeutic endoscopy, the ablation energy delivery tool is withdrawn through the flexible sheath, the flexible sheath is withdrawn through the endoscope, and the endoscope is withdrawn from the subject.

Such flexible sheaths used as guides for tool placement need to be very flexible in order to navigate through peripheral locations that may include tortuous paths, especially in bronchoscopic cases. However, determining the location and orientation of the flexible sheath is difficult to determine or confirm. The flexible sheaths described herein are for use in a variety of medical procedures, including but not limited to, endoscopic procedures, endoluminal procedures, endovascular procedures, cardiac procedures, etc.

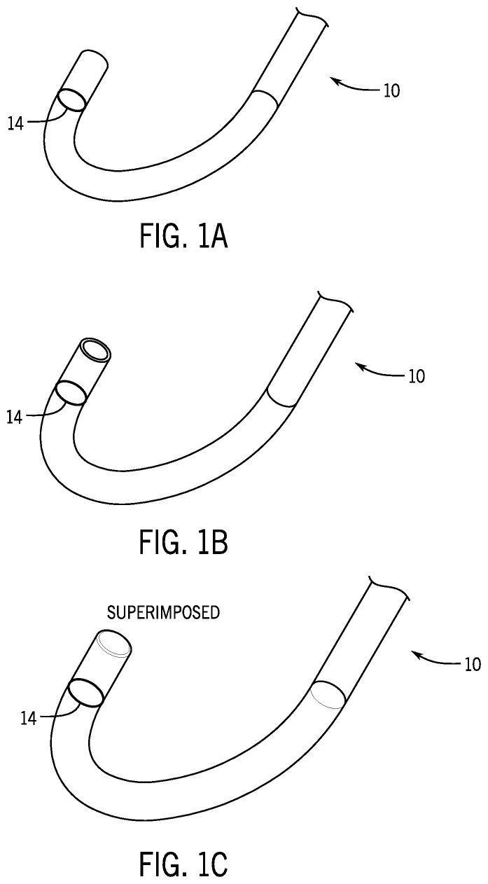

With reference to FIGS. 1A-1C, a flexible sheath 10 in two different positions and orientations (FIG. 1A and FIG. 1B) appears similar to or identical in a two-dimensional image of the flexible sheath 10 (FIG. 1C). In other words, two-dimensional images of the flexible sheath 10 in different positions and orientations can be ambiguous as to the three-dimensional position and orientation of the flexible sheath. Furthermore, a first cross-section 14 of the flexible sheath 10 in a first orientation (e.g., facing away) (FIG. 1A) and in a second orientation (e.g., facing towards) (FIG. 1B) can appear the same in a two-dimensional image (FIG. 1C). As such, conventional two-dimensional imaging of a flexible sheath in three-dimensional space can lead to ambiguity and errors.

Accordingly, new flexible sheaths capable of being localized (i.e., determining position and orientation) by new methods in real-time based on imaging (e.g., x-ray images) are needed.

The present invention addresses this need through providing fiducials capable of being localized in a two-dimensional x-ray image. Such flexible sheath assemblies are configured for use in any kind of endoscopic or endovascular procedure (e.g., tissue ablation, resection, cautery, vascular thrombosis, treatment of cardiac arrhythmias and dysrhythmias, electrosurgery, tissue harvest, etc.). The flexible sheaths described herein are for use in a variety of medical procedures, including but not limited to, endoscopic procedures, endoluminal procedures, endovascular procedures, cardiac procedures, etc.

The flexible sheaths of the present invention are not limited to particular size dimensions. Indeed, in some embodiments, the size dimension of the flexible sheath is such that it is able to fit within and pass through the lumen of a primary catheter (e.g., an endoscope). In some embodiments, the flexible sheath is of sufficient diameter (e.g., 1 mm . . . 2 mm . . . 3 mm . . . 4 mm . . . 5 mm) to accommodate within and through its interior one or more suitable tools (e.g., energy delivery device, steerable navigation catheter). In some embodiments, the flexible sheath is of sufficient length to extend from an insertion site (e.g., mouth, incision into body of subject, etc.) to a desired target region within a living body (e.g., 50 cm . . . 75 cm . . . 1 m . . . 1.5 m . . . 2 m . . . 10 m . . . 25 m, etc.). In some embodiments, the flexible sheath is of sufficient length to extend through and beyond the reach of a primary catheter (e.g., endoscope) to reach a treatment site (e.g., peripheral lung tissue, heart tissue, gastrointestinal tissue, etc.) (e.g., any desired location within a living body).

The flexible sheaths of the present invention are not limited to a particular manner of navigation through a primary catheter and/or through a body region. In some embodiments, the flexible sheath comprises a navigation and/or steering mechanism. In some embodiments, the flexible sheath is without an independent means of navigation, position recognition, or maneuvering. In some embodiments, the flexible sheath relies upon the primary catheter (e.g., endoscope) or a steerable navigation catheter for placement.

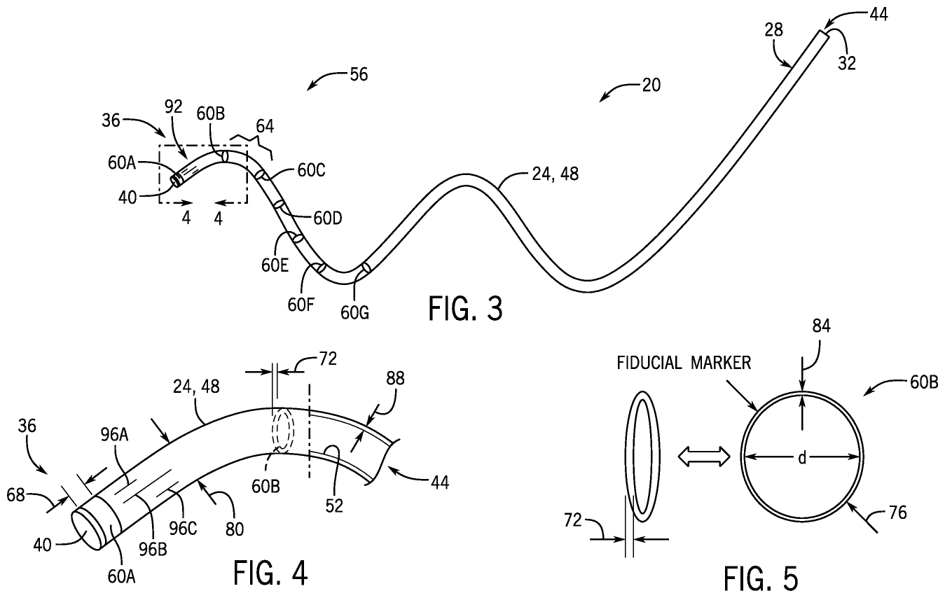

With reference to FIG. 3, a flexible sheath 20 according to one embodiment is illustrated. The flexible sheath 20 is not limited to a particular design or configuration. In some embodiments, the design or configuration of the flexible sheath 20 is such that it is able to be positioned at a desired tissue region and maintain that desired positioning during medical procedures involving use insertion and withdrawal of medical tools through the flexible sheath 20. In some embodiments, the flexible sheath 20 has sufficient flexibility to access a circuitous route through a subject (e.g., through a branched structure, through a bronchial tree, through any region of the body to reach a desired location).

With continued reference to FIG. 3, the flexible sheath 20 has an elongate tubular body 24 with an elongate tubular body proximal end 28 having a proximal end opening 32, an elongate tubular body distal end 36 having a distal end opening 40, an elongate tubular body interior portion 44 extending from the elongate tubular body proximal end 28 to the elongate tubular body distal end 36, and an elongate tubular body exterior portion 48 extending from the elongate tubular body proximal end 28 to the elongate tubular body distal end 36. In some embodiments, the arrangement and positioning of the elongate tubular body proximal end 28, proximal end opening 32, elongate tubular body distal end 36, distal end opening 40, elongate tubular body interior portion 44, and elongate tubular body exterior portion 48 within the elongate tubular body 24 is not limited. In some embodiments, the arrangement and positioning of the elongate tubular body proximal end 28, proximal end opening 32, elongate tubular body distal end 36, distal end opening 40, elongate tubular body interior portion 44, and elongate tubular body exterior portion 48 within the elongate tubular body 24 is such that it renders the flexible sheath 20 capable of being positioned at a desired tissue region and maintaining that desired positioning during medical procedures involving use insertion and withdrawal of medical tools through the flexible sheath 20.

With continued reference to FIG. 1, the elongate tubular body 24 is not limited to a particular composition. In some embodiments, the composition of the elongate tubular body 24 is any composition that renders the flexible sheath 20 capable of being positioned at a desired tissue region and maintaining that desired positioning during medical procedures involving use insertion and withdrawal of medical tools through the flexible sheath 20. In some embodiments, the composition of the elongate tubular body 24 is a polymer material. In some embodiments, the composition of the elongate tubular body 24 is a higher temperature rated polymer material. Such embodiments are not limited to a particular higher temperature rated polymer material. In some embodiments, the higher temperature rated polymer material is fluorinated ethylene propylene (FEP). In some embodiments, the higher temperature rated polymer material is a thermoplastic copolyester. In some embodiments, the thermoplastic copolyester is Arnitel. In some embodiments, the higher temperature rated polymer material is a fluoropolymer. Such embodiments are not limited to a particular fluoropolymer. In some embodiments, the fluoropolymer is perfluoromethylalkoxy alkane (MFA). In some embodiments, the fluoropolymer is perfluoroalkoxy alkane (PFA). In some embodiments, only a portion (5%, 10%, 25%, 50%, 75%, 77%, 79%, 85%, 88%, 90%, 94%, 98%, 99%, 99.999%) of the elongate tubular body 24 has a composition of a higher temperature rated polymer material. In some embodiments, only a portion (5%, 10%, 25%, 50%, 75%, 77%, 79%, 85%, 88%, 90%, 94%, 98%, 99%, 99.999%) starting from the elongate tubular body distal end 36 has a composition of a higher temperature rated polymer material. In some embodiments, the entire elongate tubular body 24 has a composition of a higher temperature rated polymer material.

With continued reference to FIG. 3, the flexible sheath 20 is configured such that devices (e.g., medical devices) can be inserted and withdrawn through the elongate tubular body interior portion 44. Examples of such devices that can be inserted and withdrawn through the elongate tubular body interior portion 44 include, but are not limited to, an obturator, ablation probe, energy delivery device, biopsy tool, etc.

With continued reference to FIG. 3, the elongate tubular body interior portion 44 is not limited to particular configuration permitting the insertion and withdrawal of devices. In some embodiments, the elongate tubular body interior portion 44 has therein a hollow port 52 extending from the proximal end opening 32, through the elongate tubular body proximal end 28, through the elongate tubular body distal end 36, and out the distal end opening 40. The hollow port 52 is not limited to a particular size. In some embodiments, the size of the hollow port 52 is such that it can accommodate the insertion and withdrawal of a properly sized device (e.g., a device having a circumference smaller than the circumference of the hollow port 52) through its entirety. In some embodiments, the size of the hollow port 52 is such that it can accommodate the insertion and withdrawal of a properly sized device (e.g., a device having a circumference smaller than the circumference of the hollow port 52) through its entirety without compromising the ability of the flexible sheath 20 to be positioned at a desired tissue region and maintaining that desired positioning during medical procedures.

With continued reference to FIG. 3, the flexible sheath 20 includes a plurality of fiducials 56 (also referred to herein as markers). As explained further herein, the plurality of fiducials 56 provides visual X-ray indication of the location and orientation of the flexible sheath 20 in three-dimensional space. In other words, the plurality of fiducials 56 enable real-time image-based localization of the position and orientation of the flexible sheath 20. The plurality of fiducials 56 include a radiopaque material. In some embodiments, the radiopaque material is barium sulfate, bismuth, gold, tantalum, platinum, platinum iridium, stainless steel, or tungsten. The radiopaque material is opaque to x-rays or similar radiation.

In the illustrated embodiment, the flexible sheath 20 includes a first fiducial 60A positioned at the elongate tubular body distal end 36, a second fiducial 60B spaced apart from the first fiducial 60A, and a third fiducial 60C spaced apart from the second fiducial 60B. In the illustrated embodiment, the second fiducial 60B is positioned between the first fiducial 60A and the third fiducial 60C. In the illustrated embodiment, the first fiducial 60A, the second fiducial 60B, and the third fiducial 60C are spaced an equal distance 64 apart from each other. The illustrated flexible sheath 20 further includes a fourth fiducial 60D, a fifth fiducial 60E, a sixth fiducial 60F, and a seventh fiducial 60G all spaced along the length of the flexible sheath, with the distance 64 between adjacent fiducials (e.g., 60B and 60C). The flexible sheath is not limited to a particular number of fiducials. In some embodiments, the flexible sheath includes any number of fiducials. In some embodiments, at least half of the length of the sheath 20 includes fiducials.

With reference to FIG. 4, the first fiducial 60A is larger than the second fiducial 60B. In the illustrated embodiment, the first fiducial 60A is a band of radiopaque material with a width 68 larger than a width 72 of the second fiducial 60B. The larger first fiducial 60A improves visualization of the elongate tubular body distal end 36. Identifying where the elongate tubular body distal end 36 is located is advantageous because it improves safety because potentially sharp instruments, for example, exit the distal opening 40 and extend from the elongate tubular body distal end 36. In other words, the first fiducial 60A is larger than the other remaining fiducials (e.g., 60B-60G) for fast and accurate visual identification of the elongate tubular body distal end 36; even when superimposed by bones or other dense body organs. Identifying where the elongate tubular body distal end 36 is located also advantageously improves the ability of an operator to retract the flexible sheath 20 back along an instrument (e.g., probe) to remove the flexible sheath 20 from an ablation zone, for example. The distal ends of conventional flexible sheaths are difficult to visualize and therefore difficult to ensure they are retracted a desired distance away from an instrument. Furthermore, the larger first fiducial 60A advantageously provides a sturdy mechanical attachment point for an articulation mechanism (e.g., an anchor for pull wires).

With reference to FIGS. 4 and 5, the second fiducial 60B is a circular marker (i.e., circular in shape, ring-shaped). In some embodiments, the second fiducial 60B wraps around a portion (e.g., 1%, 5%, 10%, 25%, 45%, 49.9%, 50%, 55%, 62%, 70%, 79.5%, 85%, 90%, 92%, 93.5%, 98%, 99%, 99.99%) of the elongate tubular body exterior portion 48. In some embodiments, the second fiducial 60B is a three-dimensional circle or ring. In some embodiments, an outer diameter 76 of the second fiducial 60B is equal to an outer diameter 80 of the elongate tubular body 24. In some embodiments, a thickness 84 of the second fiducial 60B is equal to a wall thickness 88 of the elongate tubular body 24. In some embodiments, all of the fiducials 60A-60G are identically sized and shaped. In other embodiments, each of the fiducials 60A, 60B, 60C, etc. is uniquely sized and/or shaped.

With continued reference to FIG. 4, the flexible sheath 20 further includes an asymmetric tip marker 92 positioned at the elongate tubular body distal end 36. In the illustrated embodiment, the asymmetric tip marker 92 is one of the plurality of fiducials 56. In the illustrated embodiment, the asymmetrical tip 92 provides visual X-ray indication of the orientation of the elongate tubular body distal end 36 in three-dimensional space. In some embodiments, the asymmetrical tip 92 provides visual X-ray indication of a pointing direction of the flexible sheath 20 (i.e., facing direction of the distal opening 40).

With continued reference to FIG. 4, the asymmetric tip marker 92 includes a first longitudinal mark 96A, a second longitudinal mark 96B circumferentially spaced from the first longitudinal mark 96A, and a third longitudinal mark 96C circumferentially spaced from the second longitudinal mark 96B. In the illustrated embodiment, the second longitudinal mark 96B is circumferentially positioned between the first longitudinal mark 96A and the third longitudinal mark 96C. In the illustrated embodiment, the second longitudinal mark 96B is longer than the first longitudinal mark 96A and longer than the third longitudinal mark 96C. In the illustrated embodiment, the first longitudinal mark 96A is positioned closer to the elongate tubular body distal end 36 than the third longitudinal mark 96C. The arrangement of the longitudinal marks 96A-96C visually indicates to a user the orientation of the flexible sheath 20. In some embodiments, the second longitudinal mark 96B is a centerline and the first longitudinal mark 96A indicates one side of the centerline and the third longitudinal mark 96C indicates the other side of the centerline.

In some embodiments, the flexible sheaths further contain a steerable pull ring. Such embodiments are not limited to a particular configuration for the steerable pull ring. In some embodiments, the steerable pull ring has any configuration that permits a user to manually steer the flexible sheath via manipulation of the steerable pull ring (e.g., manipulation of one or both of the wires results in a curving or steering of the sheath). In some embodiments, the asymmetric tip marker 92 provides visual X-ray indication of an articulation axis of the flexible sheath. In other words, the asymmetric tip marker 92 indicates to a user in a two-dimensional image which direction the flexible sheath will articulate when the steerable pull ring is utilized.

In some embodiments, the steerable pull ring permits the flexible sheath to be steered in any desired manner or direction. For example, in some embodiments, the steerable pull ring permits the flexible sheath to be steered at any desired curve angle (e.g., from 1 to 180 degrees). In some embodiments, the steerable pull ring permits the flexible sheath to be steered at any desired bend angle (e.g., from 1 to 360 degrees). In some embodiments, the steerable pull ring permits the flexible sheath to be steered at any desired bend radius (e.g., from 1 to 360 degrees). In some embodiments, the steerable pull ring permits the flexible sheath to be steered at any desired curve diameter. In some embodiments, the steerable pull ring permits the flexible sheath to be steered at any desired reach (e.g., from 0.1 to 100 mm). In some embodiments, the steerable pull ring permits the flexible sheath to be steered at any desired curl. In some embodiments, the steerable pull ring permits the flexible sheath to be steered at any desired sweep. In some embodiments, the steerable pull ring permits the flexible sheath to be steered at any desired curve (e.g., symmetrical or asymmetrical) (e.g., multi-curve or compound curve). In some embodiments, the steerable pull ring permits the flexible sheath to be steered at any desired loop. In some embodiments, the steerable pull ring permits the flexible sheath to be steered at any desired deflection (e.g., on-plane deflection, off plane deflection).

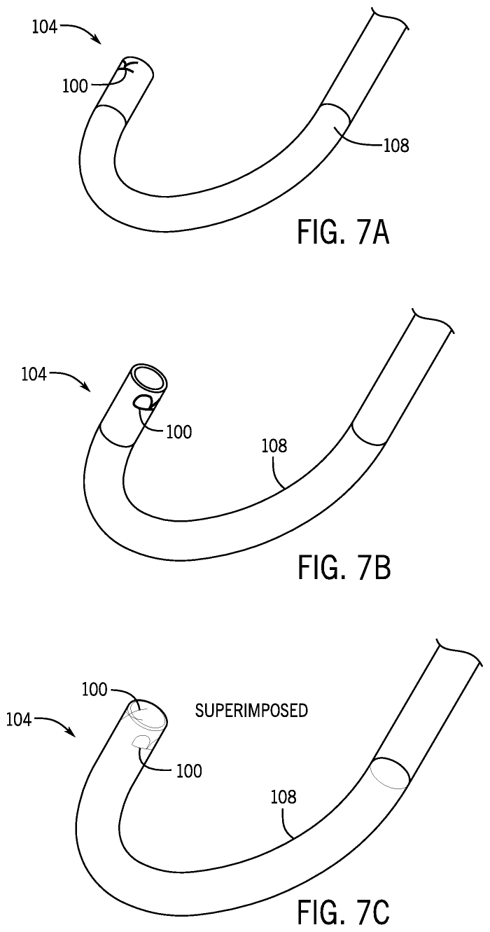

With reference to FIG. 7A-7C, an asymmetric tip marker 100 according to another embodiment is illustrated positioned at an elongate tubular body distal end 104 of a flexible sheath 108. The illustrated embodiment, the asymmetric tip marker 100 is in the shape of an “R.” In some embodiments, the “R” indicates a designated “right-side” of the flexible sheath 108), which provides visual indication the orientation of the flexible sheath 108. In some embodiments, the asymmetric tip marker 100 provides visual indication of an articulation axis of the elongate tubular body distal end 104.

With continued reference to FIGS. 7A-7C, the flexible sheath 108 in two different positions and orientations (FIG. 7A and FIG. 7B) is distinguishable in a two-dimensional image of the flexible sheath 108 (FIG. 7C). In other words, two-dimensional images of the flexible sheath 108 in different positions and orientations are unique and not ambiguous, resulting in unambiguous visual indication of the three-dimensional position and orientation of the flexible sheath 108. As such, the asymmetric tip marker 100 advantageously reduces the ambiguity and error in localizing (i.e., determining position and orientation) the flexible sheath 108 in three-dimensional space based on two-dimensional imaging.

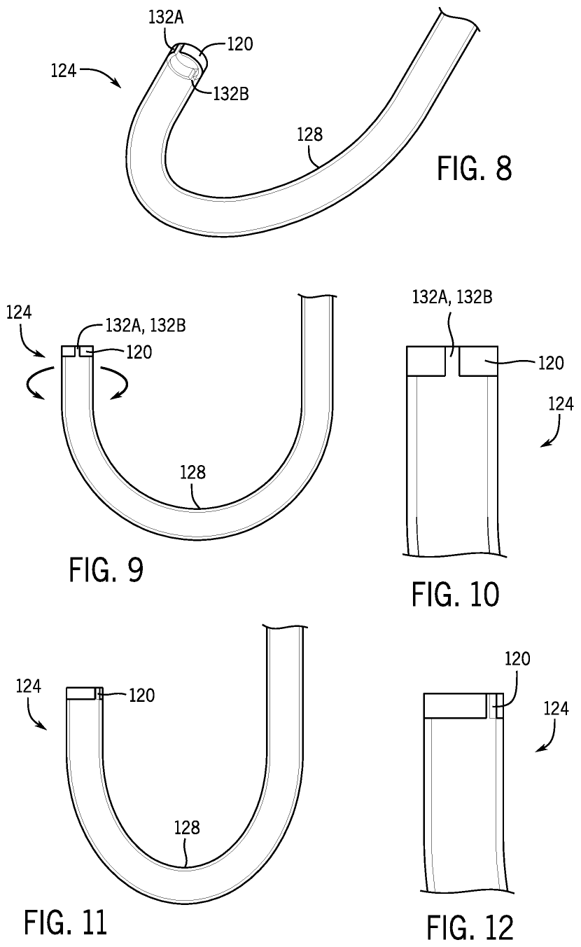

With reference to FIGS. 8-12, an asymmetrical tip marker 120 according to another embodiment is illustrated positioned at an elongate tubular body distal end 124 of a flexible sheath 128. The asymmetrical tip marker 120 is a ring positioned over the elongate tubular body distal end 124. The ring 120 includes a first notch 132A and a second notch 132B. With reference to FIGS. 9 and 10, the flexible sheath 128 is oriented such that articulation of the elongate tubular body distal end 124 occurs within the plane of view. In contrast, with reference to FIGS. 11 and 12, the flexible sheath 128 is oriented such that the articulation of the elongate tubular body distal end 124 occurs obliquely to the plane of view. The orientation of FIGS. 9 and 10 advantageously indicates how the elongate tubular body distal end 124 is oriented and allows an operator to visualize in two-dimensions the articulation of the flexible sheath 128.

In the illustrated embodiment, the first notch 132A and the second notch 132B align with each other when the elongate tubular body distal end 124 is oriented such that articulation is in the plane of view. Alignment of the first notch 132A and the second notch 132B (FIG. 10) results in a gap visually shown in the two-dimensional image of the radiopaque ring 120. As such, an operator may manipulate the flexible sheath 128 until the asymmetrical tip marker 120 provides indication that articulation of the flexible sheath 128 will occur within the viewing plane of the two-dimensional image.

In some embodiments, the present invention provides systems for therapeutic endoscopic procedures wherein flexible sheaths as described herein, primary catheters, and one or more suitable tools (e.g., energy delivery device, steerable navigation catheter) are provided.

Such embodiments are not limited to a particular type or kind of primary catheter. In some embodiments, the present invention primary catheter is an endoscope. In some embodiments, any suitable endoscope known to those in the art finds use as a primary catheter in the present invention. In some embodiments, a primary catheter adopts characteristics of one or more endoscopes and/or bronchoscopes known in the art, as well as characteristics described herein. One type of conventional flexible bronchoscope is described in U.S. Pat. No. 4,880,015, herein incorporated by reference in its entirety. The bronchoscope measures 790 mm in length and has two main parts, a working head and an insertion tube. The working head contains an eyepiece; an ocular lens with a diopter adjusting ring; attachments for suction tubing, a suction valve, and light source; and an access port or biopsy inlet, through which various devices and fluids can be passed into the working channel and out the distal end of the bronchoscope. The working head is attached to the insertion tube, which typically measures 580 mm in length and 6.3 mm in diameter. The insertion tube contains fiberoptic bundles, which terminate in the objective lens at the distal tip, light guides, and a working channel. Other endoscopes and bronchoscopes which may find use in embodiments of the present invention, or portions of which may find use with the present invention, are described in U.S. Pat. Nos. 7,473,219; 6,086,529; 4,586,491; 7,263,997; 7,233,820; and 6,174,307.

Such embodiments are not limited to a particular type or kind of steerable navigation catheter. In some embodiments, a steerable navigation catheter is configured to fit within the lumen of a primary catheter (e.g., endoscope) and a flexible sheath. In some embodiments, a steerable navigation catheter is of sufficient length to extend from an insertion site (e.g. mouth, incision into body of subject, etc.) to a treatment site (e.g. 50 cm . . . 75 cm . . . 1 m . . . 1.5 m . . . 2 m . . . 5 m . . . 15 m). In some embodiments, a channel catheter is of sufficient length to extend beyond the reach of a primary catheter (e.g., endoscope) to reach a treatment site (e.g. peripheral lung tissue). In some embodiments, a steerable navigation catheter engages a flexible sheath such that movement of the steerable navigation catheter results in synchronous movement of the flexible sheath. In some embodiments, as a steerable navigation catheter is inserted along a path in a subject, the flexible sheath surrounding the steerable navigation catheter moves with it. In some embodiments, a flexible sheath is placed within a subject by a steerable navigation catheter. In some embodiments, a steerable navigation catheter can be disengaged from a flexible sheath. In some embodiments, disengagement of a steerable navigation catheter and flexible sheath allows movement of the steerable navigation catheter further along a pathway without movement of the flexible sheath. In some embodiments, disengagement of a steerable navigation catheter and flexible sheath allows retraction of the steerable navigation catheter through the flexible sheath without movement of the flexible sheath.

Such embodiments are not limited to a particular type or kind of energy delivery device (e.g., ablation device, surgical device, etc.) (see, e.g., U.S. Pat. Nos. 7,101,369, 7,033,352, 6,893,436, 6,878,147, 6,823,218, 6,817,999, 6,635,055, 6,471,696, 6,383,182, 6,312,427, 6,287,302, 6,277,113, 6,251,128, 6,245,062, 6,026,331, 6,016,811, 5,810,803, 5,800,494, 5,788,692, 5,405,346, 4,494,539, U.S. patent application Ser. Nos. 11/728,460, 11/728,457, 11/728,428, 11/237,136, 11/236,985, 10/980,699, 10/961,994, 10/961,761, 10/834,802, 10/370,179, 09/847,181; Great Britain Patent Application Nos. 2,406,521, 2,388,039; European Patent No. 1395190; and International Patent Application Nos. WO 06/008481, WO 06/002943, WO 05/034783, WO 04/112628, WO 04/033039, WO 04/026122, WO 03/088858, WO 03/039385 WO 95/04385; each herein incorporated by reference in their entireties). Such energy delivery devices are not limited to emitting a particular kind of energy. In some embodiments, the energy delivery devices are capable of emitting radiofrequency energy. In some embodiments, the energy delivery devices are capable of emitting microwave energy. Such devices include any and all medical, veterinary, and research applications devices configured for energy emission, as well as devices used in agricultural settings, manufacturing settings, mechanical settings, or any other application where energy is to be delivered.

The systems for therapeutic endoscopic procedures of the present invention are not limited to particular uses. Indeed, such systems of the present invention are designed for use in any setting wherein the emission of energy is applicable. Such uses include any and all medical, veterinary, and research applications. In addition, the systems and devices of the present invention may be used in agricultural settings, manufacturing settings, mechanical settings, or any other application where energy is to be delivered.

In some embodiments, the systems are configured for any type of procedure wherein the flexible sheath described herein can find use. For example, the systems find use for open surgery, percutaneous, intravascular, intracardiac, intraluminal, endoscopic, laparoscopic, or surgical delivery of energy.

The present invention is not limited by the nature of the target tissue or region. Uses include, but are not limited to, treatment of heart arrhythmia, tumor ablation (benign and malignant), control of bleeding during surgery, after trauma, for any other control of bleeding, removal of soft tissue, tissue resection and harvest, treatment of varicose veins, intraluminal tissue ablation (e.g., to treat esophageal pathologies such as Barrett's Esophagus and esophageal adenocarcinoma), treatment of bony tumors, normal bone, and benign bony conditions, intraocular uses, uses in cosmetic surgery, treatment of pathologies of the central nervous system including brain tumors and electrical disturbances, sterilization procedures (e.g., ablation of the fallopian tubes) and cauterization of blood vessels or tissue for any purposes. In some embodiments, the surgical application comprises ablation therapy (e.g., to achieve coagulative necrosis). In some embodiments, the surgical application comprises tumor ablation to target, for example, metastatic tumors. In some embodiments, the systems including the flexible sheath described herein are configured for movement and positioning, with minimal damage to the tissue or organism, at any desired location, including but not limited to, the lungs, brain, neck, chest, abdomen, and pelvis. In some embodiments, the systems are configured for guided delivery, for example, by computerized tomography, ultrasound, magnetic resonance imaging, fluoroscopy, and the like. Indeed, in some embodiments, all inserted components of such a system are configured for movement along a narrow and circuitous path through a subject (e.g. through a branched structure, through the bronchial tree, etc.).

In certain embodiments, the present invention provides methods of treating a tissue region, comprising providing a tissue region and a system described herein (e.g., a primary catheter (e.g., an endoscope), a flexible sheath as described herein, and an energy delivery device (e.g., a microwave ablation catheter), and at least one of the following components: a processor, a power supply, a temperature monitor, an imager, a tuning system, a temperature reduction system, and/or a device placement system); positioning a portion of the energy delivery device in the vicinity of the tissue region, and delivering an amount of energy with the device to the tissue region. In some embodiments, the tissue region is a tumor. In some embodiments, the delivering of the energy results in, for example, the ablation of the tissue region and/or thrombosis of a blood vessel, and/or electroporation of a tissue region. In some embodiments, the tissue region is a tumor. In some embodiments, the tissue region comprises one or more of the lung, heart, liver, genitalia, stomach, lung, large intestine, small intestine, brain, neck, bone, kidney, muscle, tendon, blood vessel, prostate, bladder, and spinal cord.

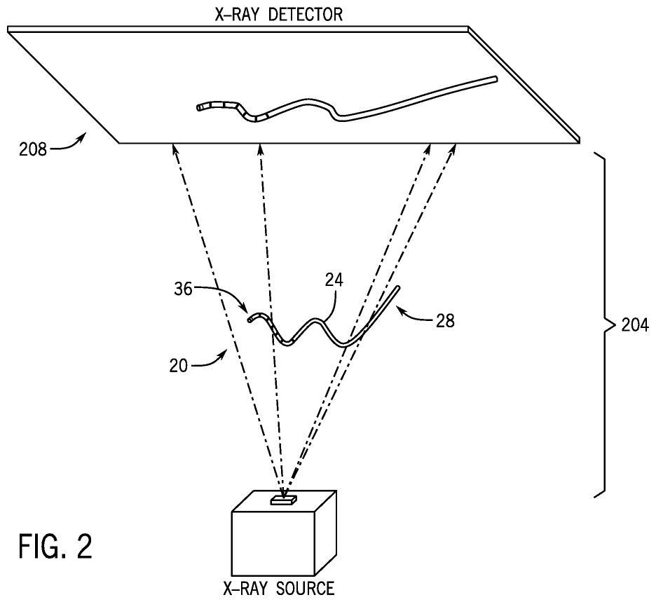

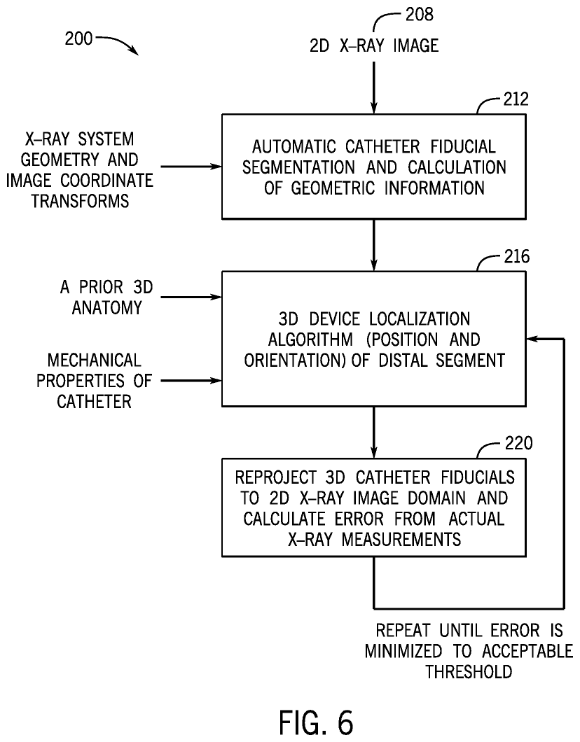

With reference to FIG. 6, the present invention provides a method 200 of localizing a flexible sheath in three-dimensional space. The method 200 includes positioning the flexible sheath with at least one radiopaque fiducial (illustrated as the flexible sheath 20) in an x-ray imaging system 204 (FIG. 2) and capturing a two-dimensional x-ray image 208 of the flexible sheath. In the illustrated embodiment, the x-ray imaging system 204 includes an x-ray source and an x-ray detector. The method 200 further includes a STEP 212 that utilizes the geometry of the x-ray imaging system 204 and corresponding image coordinate transforms to perform automatic fiducial segmentation and calculation of geometric information. In other words, STEP 212 includes identifying the at least one fiducial in the two-dimensional x-ray image 208.

With continued reference to FIG. 6, the method 200 further includes a STEP 216 that utilizes a three-dimensional device localization algorithm to localize (i.e., determine position and orientation) of a portion of the flexible sheath (e.g., the distal end portion). In other words, STEP 216 includes determining an estimated location of the flexible sheath based on a geometric transform of the x-ray imaging system 204. In some embodiments, the three-dimensional device localization algorithm is adapted for lung anatomy and/or a steerable catheter. In some embodiments, the localization algorithm includes one or more of the following approaches: (1) Epipolar based reconstruction from multiple X-ray view angles (see Kalmykova, M 2018, ‘An approach to point-to-point reconstruction of 3D structure of coronary arteries from 2D X-ray angiography, based on epipolar constraints’, 7^{th} International Young Scientist Conference on Computational Science; and Brost, A 2009, ‘Accuracy of x-ray image-based 3D localization from two C-arm views: A comparison between an ideal system and a real device’, Proceedings of SPIE—The International Society for Optical Engineering); (2) Epipolar recon combined with known device properties (see Vernikouskaya, I 2021, ‘Cyro-balloon catheter localization in X-Ray fluoroscopy using U-net’, International Journal of Computer Assisted Radiology and Surgery, 16:1255-1262); (3) Machine learning based device-specific pose detection from a single X-ray (see Ralovhich, K 2014, ‘6 DoF Catheter Detection, Application of Intracardiac Echocardiography’, Springer International Publishing Switzerland; and Hatt, C 2016, ‘Real-time pose estimation of device from x-ray images: Application to x-ray/echo registration for cardiac interventions’, Med Image Anal. 34:101-108); and/or (4) Anatomical constrained reconstruction to constrain the localization algorithm of the device to a specific segmented anatomical region (see Mandal, K 2016, ‘Vessel-based registration of an optical shape sensing catheter for MR navigation’, Int J CARS, 11:1025-1034).

In some embodiments, the STEP 216 further includes displaying the estimated location of the flexible sheath in real-time on a display. In some embodiments, the determining of the estimated location of the flexible sheath is further based on three-dimensional anatomical constraints of a patient (i.e., a priori three-dimensional anatomical information). For example, the centerlines of the pulmonary tree in three-dimensional space is segmented and used to constrain the device localization from X-ray as being bounded to some limited distance on or way from the center line of anatomy. The anatomical constraints are especially useful for objects that are static of within a known motion or deformation. In other embodiments, the determining of the estimated location of the flexible sheath is further based on a mechanical property (e.g., continuous lumen, geometrical constraints, rigidity and compressibility) of the flexible sheath.

With continued reference to FIG. 6, the method 200 further includes a STEP 220 that includes validating the estimated location of the flexible sheath by reprojecting the estimated location of the fiducial(s) into a two-dimensional validation image and calculating an error between the location of the fiducial(s) in the two-dimensional x-ray image 208 and the two-dimensional validation image. In other words, STEP 220 includes reprojecting the estimated three-dimensional fiducial locations to the two-dimensional x-ray image domain and calculating the error compared to the actual x-ray measurements 208. In some embodiments, STEPS 216 and 220 are repeated until the resulting error is below a threshold (e.g., acceptable level).

Other localization methods which may find use in embodiments of the present invention, or portions of which may find use with the present invention, are described in U.S. Pat. No. 9,232,924; International Patent Application No WO2017/070205; and U.S. Patent Application Publication Nos. US2017/0319165 US2006/0233423; US2011/0282151; and US2017/0358091—each of which is incorporated herein by reference in their entireties.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method of localizing a flexible sheath in three-dimensional space, the method comprising:
positioning the flexible sheath with at least one fiducial in an x-ray imaging system;
capturing a two-dimensional x-ray image of the flexible sheath;
identifying the at least one fiducial in the two-dimensional x-ray image; and
determining an estimated location of the flexible sheath based on a geometric transform of the x-ray imaging system.

2. The method of claim 1, wherein determining the estimated location of the flexible sheath is further based on three-dimensional anatomical constraints of a patient.

3. The method of claim 2, wherein three-dimensional anatomical constraints include centerlines of a pulmonary tree in three-dimensional space.

4. The method of claim 1, wherein determining the estimated location of the flexible sheath is further based on a mechanical property of the flexible sheath.

5. The method of claim 4, wherein the mechanical property is continuity, rigidity, or compressibility.

6. The method of claim 1, further comprising validating the estimated location of the flexible sheath by reprojecting the estimated location of the at least one fiducial into a two-dimensional validation image, and calculating an error between the location of the at least one fiducial in the two-dimensional x-ray image and the two-dimensional validation image.

7. The method of claim 6, wherein the determining of the estimated location is repeated until the error is below a threshold.

8. The method of claim 1, further comprising displaying the estimated location of the flexible sheath in real-time.

9. The method of claim 1, further comprising determining an estimated orientation of the flexible sheath based on the at least one fiducial.

10. The method of claim 1, wherein determining the estimated location of the flexible sheath utilizes Epipolar reconstruction.

11. The method of claim 1, wherein capturing the two-dimensional x-ray image is from a first angle and the method further includes capturing a two-dimensional x-ray image from a second angle different than the first angle.

12. The method of claim 1, wherein the at least one fiducial includes a first fiducial, a second fiducial, and a third fiducial, wherein the second fiducial is positioned between the first fiducial and the third fiducial.

13. The method of claim 12, wherein the first fiducial, the second fiducial, and the third fiducial are spaced an equal distance apart from each other.

14. The method of claim 12, wherein the first fiducial is circular.

15. The method of claim 1, wherein the at least one fiducial includes an asymmetric tip marker aligned to an articulation axis of the flexible sheath.

16. The method of claim 15, wherein the asymmetrical tip includes a first longitudinal mark, a second longitudinal mark circumferentially spaced from the first longitudinal mark, and a third longitudinal mark circumferentially spaced from the second longitudinal mark, the second longitudinal mark is circumferentially positioned between the first longitudinal mark and the third longitudinal mark.

17. The method of claim 16, wherein the second longitudinal mark is longer than the first longitudinal mark and the third longitudinal mark.
